(19) United States
(12) Patent Application Publication
(10) Pub. No.: US 2016/0048642 A1
(43) Pub. Date: Feb. 18, 2016

(54) REAL-TIME DEMAND CAPACITY AND PATIENT THROUGHPUT MANAGEMENT DASHBOARD

(71) Applicants: THE JOHNS HOPKINS UNIVERSITY, Baltimore, MD (US); JOHN HOPKINS HEALTH SYSTEMS CORPORATION, Baltimore, MD (US)

(72) Inventors: Douglas Brooks, Baltimore, MD (US); Hetal Rupani, Columbia, MD (US)

(21) Appl. No.: 14/436,494

(22) PCT Filed: Oct. 18, 2013
(86) PCT No.: PCT/US2013/065596

§ 371 Date: Apr. 17, 2015

Related U.S. Application Data
(60) Provisional application No. 61/715,505, filed on Oct. 18, 2012.

(51) Int. Cl. G06F 19/00; G06Q 50/22; G06Q 10/06

(52) U.S. Cl. CPC G06F 19/327; G06Q 10/06313; G06Q 50/22

(57) ABSTRACT

An embodiment in accordance with the present invention provides a real-time dashboard for proving a user within a department of medicine with real-time information regarding availability and demand for hospital beds throughout a department of medicine. The dashboard can be used to track patients from a point of entry to occupation of a bed. The dashboard also tracks needs from different points of admission such as the emergency department. The user can view the status of beds and the data can be further segmented by a level of care needed by the patient. The needs and preferences of different departments within the main department of medicine can be integrated with the availability and demand information provided by the dashboard. An alert system can also be implemented using the dashboard in order to allow the user to identify bottlenecks in the placement of patients in appropriate hospital beds.

### CROSS REFERENCE TO RELATED APPLICATIONS

This application claims the benefit of U.S. Provisional Patent Application No. 61/715,505 filed on Oct. 18, 2012, which is incorporated by reference, herein, in its entirety.

### FIELD OF THE INVENTION

The present invention relates generally to hospital management. More particularly the present invention relates to bed management for hospital patients.

### BACKGROUND OF THE INVENTION

A typical large hospital sees over 13,000 inpatients per year, runs about 220 beds, employs nearly 600 nurses, and works with over 100 individual physicians. The department of medicine, at such an institution, can receive inpatients from a variety of locations. The emergency room department (ED) can also supply between 60% and 65% of the Department's inpatients, and the remaining come from the admitting department, and various procedural units such as Endoscopy, Interventional Pulmonary, and the Cardiology/Vascular Diagnostic Lab. Discharge from the department of medicine is also a complex matter, with patients being discharged to home, other hospitals, rehab facilities (short term and long term), and skilled nursing facilities.

The patient population at such an institution can also range from the inner city urbanite to international patients. Traditionally, a department will manage patient flow into and out of the department in a very disjointed and siloed manner. Administratively, the financial and operations functions have reviewed data on an aggregated and averaged basis usually in the form of annual, quarterly and monthly reviews. This results in a serious time lag between actual patient flow and the decision making process of reacting to interruptions, break downs, and changes to that flow. As an example, if the November data shows a dramatic change in ED volumes coming into the department of medicine, this data is not reviewed by decision makers and planners until late December. Action plans may not be developed, reviewed and implemented for another month. By this time, two more months have passed by and the plan may no longer be relevant to the current situation, nor would a plan reacting to November volumes necessarily be relevant to February or March volumes.

Seasonalization of volumes greatly effect volumes, since a majority of admissions are emergent or urgent. In an attempt to reduce the time lag between current issues and planning, it would be advantageous for a department of medicine to be able to close the gap. In one possible solution, reports can be generated on a weekly basis or a daily basis. However, these reviews are still retrospective in nature and do not always reach the true decisions makers such as nursing management and physicians in a timely manner. Another solution includes using a strategy of averaging the deployment of resources over time and planning for the “worst case scenario” by fully staffing beds regardless of whether demand was high or not. Unfortunately, this solution can easily result in wasted resources.

Additionally, the communication process between the department of medicine and the ED can be a reactive process with each side knowing their business but no real or valuable understanding of the issues faced by the other. To some lesser extent, this breakdown in communication also can occur between the admitting office and the procedural areas. As an example, the Cardiology lab can have no real knowledge of bed supply during the days of the week and may schedule procedures based on physician preference and their own issues without taking into any account the variation in demand from other areas such as the ED or Admitting.

It would therefore be advantageous to provide a mechanism by which patient flow could be monitored on a real time basis and that this data could be distributed to all stakeholders so that each responsible party would be able to see the same data at the same time. The value proposition would be that with transparent, timely and relevant data, the traditionally siloed parties would be able to make real time decisions and engage in a more collaborative process of workflow and process change.

### SUMMARY OF THE INVENTION

The foregoing needs are met, to a great extent, by the present invention, wherein in one aspect a method for a system for presenting availability and demand for hospital beds within a department of medicine to a user includes a server configured to store data related to hospital bed availability and hospital bed demand. The system also includes a computing device configured to communicate with the server. The computing device further is in communication with a non-transitory computer readable medium programmed to execute steps including tracking a patient from a point of entry of the patient to a point of hospital bed occupation to determine demand for the hospital bed. The non-transitory computer readable medium can also track hospital bed availability within the department of medicine and display the demand and availability for the hospital beds to the user.

In accordance with another aspect of the present invention, a method for presenting availability and demand for hospital beds within a department of medicine to a user includes using a computing device configured to communicate with a server configured to store data related to hospital bed availability and hospital bed demand. The computing device is in communication with a non-transitory computer readable medium programmed for tracking a patient from a point of entry of the patient to a point of hospital bed occupation to determine demand for the hospital bed. Additionally, the non-transitory computer readable medium is programmed for tracking hospital bed availability within the department of medicine and displaying the demand and availability for the hospital beds to the user.

### BRIEF DESCRIPTION OF THE DRAWINGS

The accompanying drawings provide visual representations, which will be used to describe more fully the representative embodiments disclosed herein and can be used by those skilled in the art to better understand them and their inherent advantages. In these drawings, like reference numerals identify corresponding elements and:

FIG. 1 illustrates a flow diagram of a method for presenting availability and demand for hospital beds within a department of medicine, according to an embodiment of the present invention.

FIG. 2 illustrates a schematic diagram of a system used to present availability and demand for hospital beds within a department of medicine, according to an embodiment of the present invention.

FIG. 3 illustrates a view of an exemplary home screen for the dashboard, according to an embodiment of the present invention.

FIGS. 4A and 4B illustrate an exemplary screen for the dashboard directed to current demand based on referral source, according to an embodiment of the present invention.

FIGS. 5A and 5B illustrate an exemplary screen for the dashboard directed to current demand based on bed type, according to an embodiment of the present invention.

FIG. 6 illustrates an exemplary screen for the dashboard directed to a five-stage demand alert system, according to an embodiment of the present invention.

FIG. 7 illustrates an exemplary screen for the dashboard directed to a predictive demand from the ED, according to an embodiment of the present invention.

FIG. 8 illustrates an exemplary screen illustrating the update time and date for the last data update, according to an embodiment of the present invention.

FIGS. 9A-9C illustrate an exemplary screen illustrating additional detail related to current demand, according to an embodiment of the present invention.

FIG. 10 illustrates an exemplary screen illustrating demand originating from the Emergency Department (ED), according to an embodiment of the present invention.

FIGS. 11A and 11B illustrate an exemplary screen accessible from the screen illustrated in FIG. 10 and illustrating ED demand details, according to an embodiment of the present invention.

FIG. 12 illustrates an exemplary screen directed to current supply of beds, according to an embodiment of the present invention.

FIG. 13 illustrates an exemplary screen directed to details of current bed supply, according to an embodiment of the present invention, and FIG. 14 illustrates a drill down screen with more narrow details of bed supply based on those shown in FIG. 13.

FIG. 15 illustrates an exemplary screen related to discharge details, according to an embodiment of the present invention.

FIG. 16 illustrates an exemplary screen directed to details of bed status, according to an embodiment of the present invention.

FIG. 17 illustrates an exemplary screen directed to details of discharge delay, according to an embodiment of the present invention.

FIG. 18 illustrates an exemplary screen for tracking patient movement, according to an embodiment of the present invention.

FIG. 19 illustrates an exemplary screen for showing impediments to patient throughput, according to an embodiment of the present invention.

FIGS. 20A and 20B illustrate an exemplary screen directed to medical team location geography, according to an embodiment of the present invention. For better, faster and efficient care coordination, the patients are placed according to medical team location geography.

FIG. 21 illustrates an exemplary screen detailing admission capacity, and

FIGS. 22A-22D illustrate exemplary screens showing admission capacity details, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a real-time dashboard for proving a user within a department of medicine with real-time information regarding availability and demand for hospital beds throughout a department of medicine. The dashboard can be used to track patients from a point of entry to occupation of a bed. The dashboard also tracks needs from different points of admission such as the emergency department. The user can view the status of beds and the data can be further segmented by a level of care needed by the patient. The needs and preferences of different departments within the main department of medicine can be integrated with the availability and demand information provided by the dashboard. An alert system can also be implemented using the dashboard in order to allow the user to identify bottlenecks in the placement of patients in appropriate hospital beds.

The real-time dashboard provides multiple stakeholders immediate access to real-time demand capacity and patient flow information across multiple locations. The tool encourages a more collaborative approach for an efficient patient throughput within a department of medicine. This dashboard is designed to satisfy varied business needs of several users involved in bed management at different levels of care. The straightforward and task specific design of this dashboard allows quick assessment of real time situations and facilitates actionable decision making with full transparency and accountability. The tool tracks the movement of patients from the point of entry into the system until the patient physically occupies the bed. Users are able to monitor real time demand of beds generated at different admission points in the hospital such as ED, Admitting, Procedure areas, outpatient clinics, and HAL line. Simultaneously they will be able to view the current supply and future availability of beds. Both views are segmented by type of beds such as floor (acute) beds, IMC beds and ICU beds. The user will be further able to drill down to specific details of bed requirements, isolation status, gender, and monitor/non-monitor beds. The supply view of beds can also be further drilled down by location, isolation status, and gender level. Users can also view the current ED volume load and project the number of expected patients arriving in the next couple of hours based on predictive model. It also calculates and reports the lag between the times patients are transferred from the ED to floor beds. The tool also tracks patients that are waiting in ED for transportation to their assigned unit or bed on the floor. An inbuilt five stage alert system helps users to recognize bottlenecks in the admissions process. The dashboard also allows users to view patient loads at Medical Team or Intern level that could potentially create hurdles for patient throughput. This dashboard can usher dramatic improvements in efficiencies with seamless integration of data from multiple source systems in a real-time environment. Furthermore, multiple players are able to view the same data simultaneously and formulate plans to improve patient experience and staff morale.

This tool helps close the gap and eliminate lengthy delays between events and decision-making to resolve breakdowns, interruptions, and issues that may affect patient flow. Multiple stakeholders viewing the data simultaneously can better prioritize their work and bring in real-time changes to the patient flow process. The tool ensures accountability by different stakeholders with improved collaboration and reactive communication to achieve common goals. Finally, it helps in reducing manual effort of calling or visiting the floor to know the status of the available beds.

The tool gathers information from a number of source systems and displays visually the information is a single dashboard. The tool provides access to electronic, real-time, interactive and actionable information in an easy to understand visual format. It is capable of drilling down to the lowest level of information at each step of the patient flow process to identify and isolate the source of problem. Multiple users are able to see and interpret information at different levels of care. It provides the ability to make real time predictions regarding ED demand and other sources using predictive models.

The tool can be utilized across various departments in a main department of medicine. It is flexible enough to accommodate any user specific or external requirements that may affect the current patient flow process and demand change in the workflow. The current dashboard also has the capability of reporting various metrics including ones defined in the future by the hospital, department of medicine or other regulating groups. Continuous efforts will be made to format this dashboard to meet user specific demands that suits their process flow.

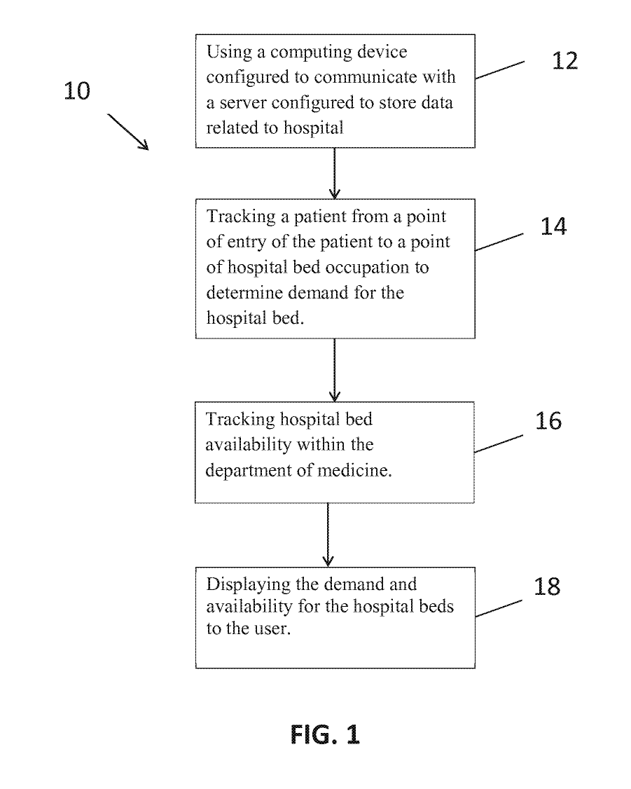

FIG. 1 illustrates a flow diagram of a method 10 for presenting availability and demand for hospital beds within a department of medicine to a user including a step 12 of using a computing device configured to communicate with a server configured to store data related to hospital bed availability and hospital bed demand. Step 14 includes using a non-transferable computer readable medium in communication with the computing device programmed for tracking a patient from a point of entry of the patient to a point of hospital bed occupation to determine demand for the hospital bed. Another step, 16, includes tracking hospital bed availability within the department of medicine. Additionally, step 18 includes displaying the demand and availability for the hospital beds to the user. The computing device can take the form of a PC, laptop, tablet, smartphone, terminal station, or any other suitable computing device known to or conceivable by one of skill in the art. The non-transitory computer readable medium can either be loaded directly onto a hard drive of the computing device, can be on a separate hard disk or CD-ROM, can be on the server described above, another independent server, a network, or any other suitable configuration known to or conceivable by one of skill in the art.

The method can also include creating a network between the server and the computing device in communication with the non-transitory computer readable medium. A user interface can be configured for the user to input information into the system as well as configured for the user to interact with the system. Another step can include tracking demand for the hospital beds from different points of admission and the different points of admission can be color coded. The demand and availability for the hospital beds can be displayed and categorized by a level of care needed by the patient. The user can also be alerted to high levels of congestion in hospital bed availability and hospital bed demand, preferably using a color coded system indicating a range from high alert to low alert. The user can also select detailed views of hospital bed availability and hospital bed demand based on specific criteria selected by the user.

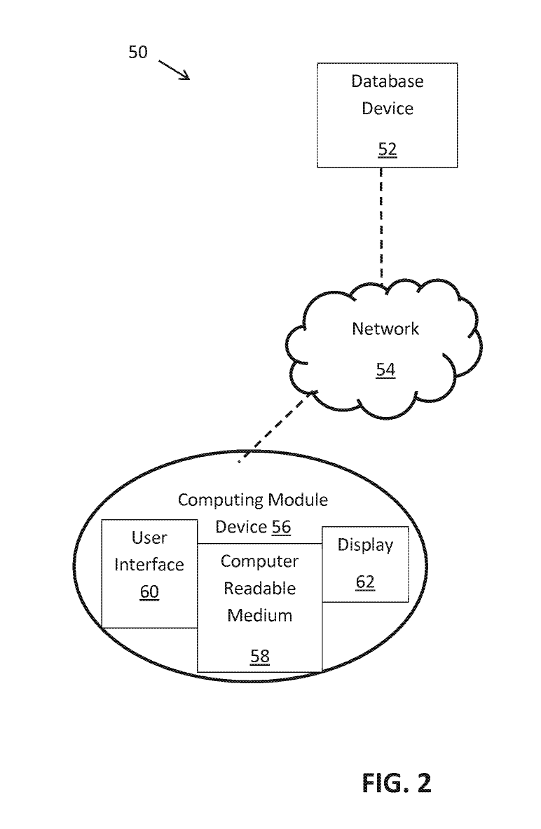

FIG. 2 illustrates a schematic diagram of the system 50 used to execute the method. The system 50 includes a database 52, and it should be noted that any suitable database known to or conceivable by one of skill in the art can be used to store and transmit the bed and demand data. The database can include information directed to availability and demand for hospital beds throughout the department of medicine. Any other suitable and relevant data can also be stored on the database. Data stored on the server 52 can be transferred via a network 54, such as a local area network, the internet, a server, or any other suitable networking construct known to or conceivable by one of skill in the art, to a computing device 56. Alternately, the computing device 56 can be a separate device connected to the imaging modality 52 using a hard wired connection.

Further, with respect to FIG. 2, the computing device 56, preferably, includes a non-transitory computer readable medium 58 or other executable disc known to one of skill in the art. The non-transitory computer readable medium can be any article of manufacture that contains data that can be read by a computer. Such computer readable media includes but is not limited to magnetic media, such as a floppy disk, a flexible disk, a hard disk, reel-to-reel tape, cartridge tape, cassette tape or cards; optical media such as CD-ROM and writeable compact disc; magneto-optical media in disc, tape or card form; and paper media, such as punched cards and paper tape. The computer readable medium contains code such that the method described herein can be executed and used to determine cardiac function. The computer readable medium 58 can also include a user interface 60 and a display 62 such that an operator can interact with the system 50 in order to input any necessary values or configure the functionality of the program as well as view information relevant to bed availability and demand by the computing device 56. The display can take the form of a computer screen, tablet computing device, smartphone, television, or other display device known to one of skill in the art. The display 62 preferably is configured such that the exemplary screens described below can be displayed. The display 62 and user interface 60 can be operated with program commands executed at least in part by the non-transitory computer readable medium 58.

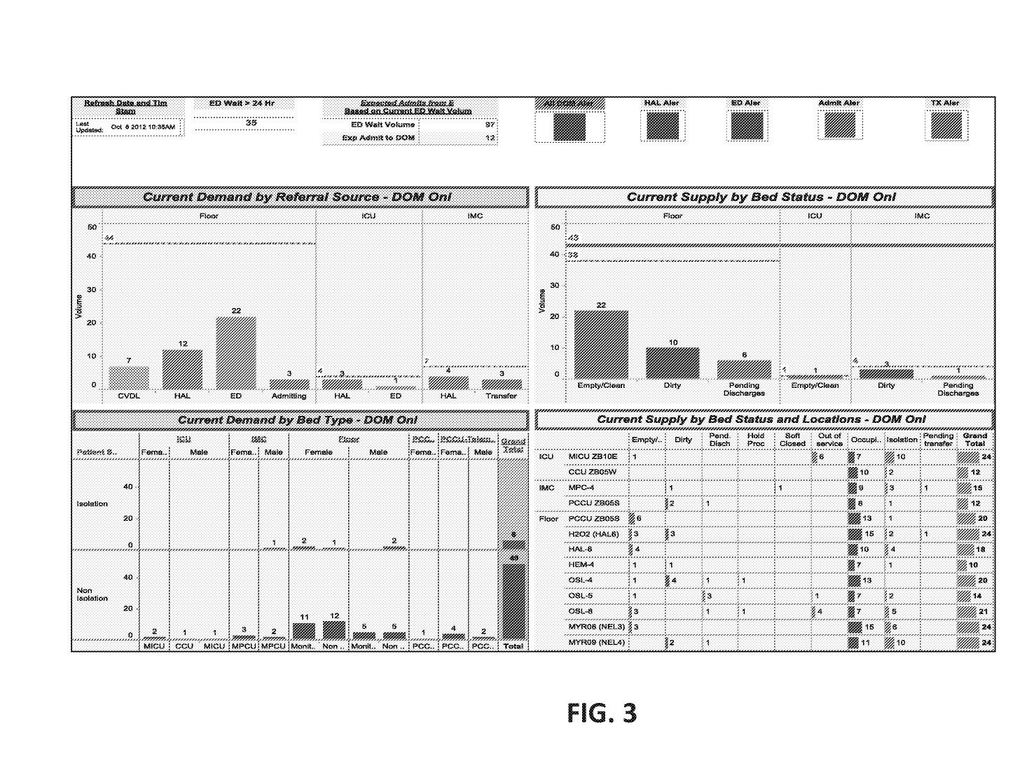

FIG. 3 illustrates a view of an exemplary home screen for the dashboard, according to an embodiment of the present invention. The home screen presents an overview of current demand and current supply organized by different criteria important to a user of the dashboard. For instance, the home screen can include graphical views of current demand by referral source, current demand by bed type, current supply by bed status, and current supply by location. All of these views can help the user to quickly understand the landscape of and challenges to placing patients in the appropriate bed. The home screen can also include metrics displayed either numerically or using color coded alerts. Exemplary metrics include a projected wait for a bed when being referred by the ED and an expected number of admits from ED along with the ED wait volume. Color coded alerts can be coded from red (high alert) to green (low alert) and can include alerts for numbers of cases waiting in a specific referral department such as the ED or alerts for the number of admits available for a given department. The home screen can also be used to drill down further with regard to any of the graphical displays of metrics shown on the home screen, using click links, a search feature, or any other suitable form of navigation known to or conceivable by one of skill in the art.

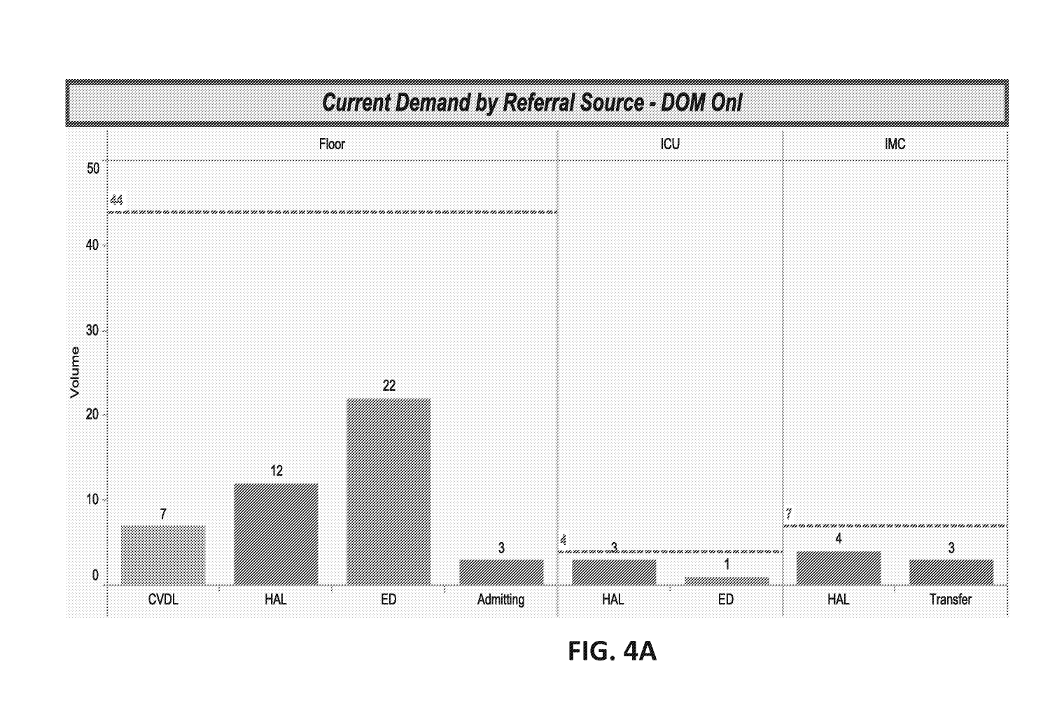

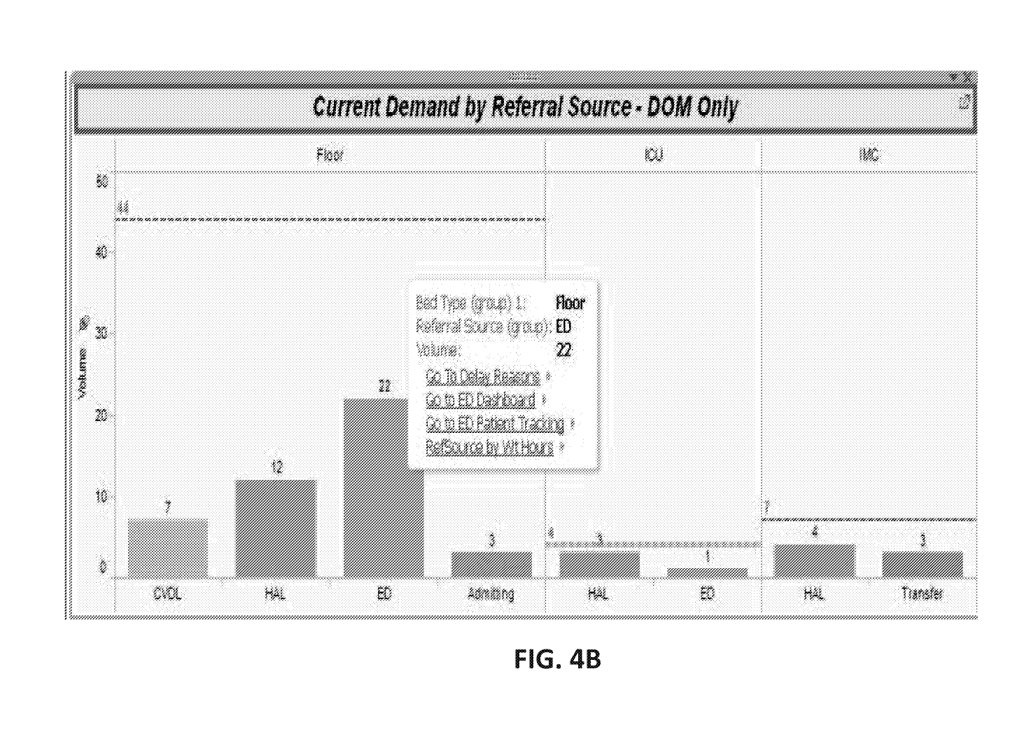

FIG. 4A illustrates an exemplary screen for the dashboard directed to current demand based on referral source, according to an embodiment of the present invention. As shown in FIG. 4A, the real-time demand for inpatient beds for the department is presented in an easy to understand graphical visuals. This information is further broken down by type of beds needed, namely, Floor, IMC and ICU beds. Demand for the beds is summed up at bed type level to easily compare demand and supply for similar type of beds. One more layer of information is shown, demand from different referral sources, namely, procedural areas, Hopkins Access Line (transfers and referral from other hospitals and facilities), Emergency Room, Admitting Office, Hopkins Outpatient Clinics, and internal transfers (and any other sources that may be defined in the source system). These referral sources are color coded which facilitates process of prioritizing admissions based on different variables such as volume, wait time and severity and diagnosis of the patients. From the same screen, as illustrated in FIG. 4B, a drill down menu is available to navigate through other dashboards. Users may choose to go to ED Dashboard to obtain more details about current situation in ED. There is an option of viewing patient level details for each referral source that will provide a view that will show total patients waiting at selected referral source by hours. Another view that can be viewed is tracking patient movement from ED to Bed Assignment.

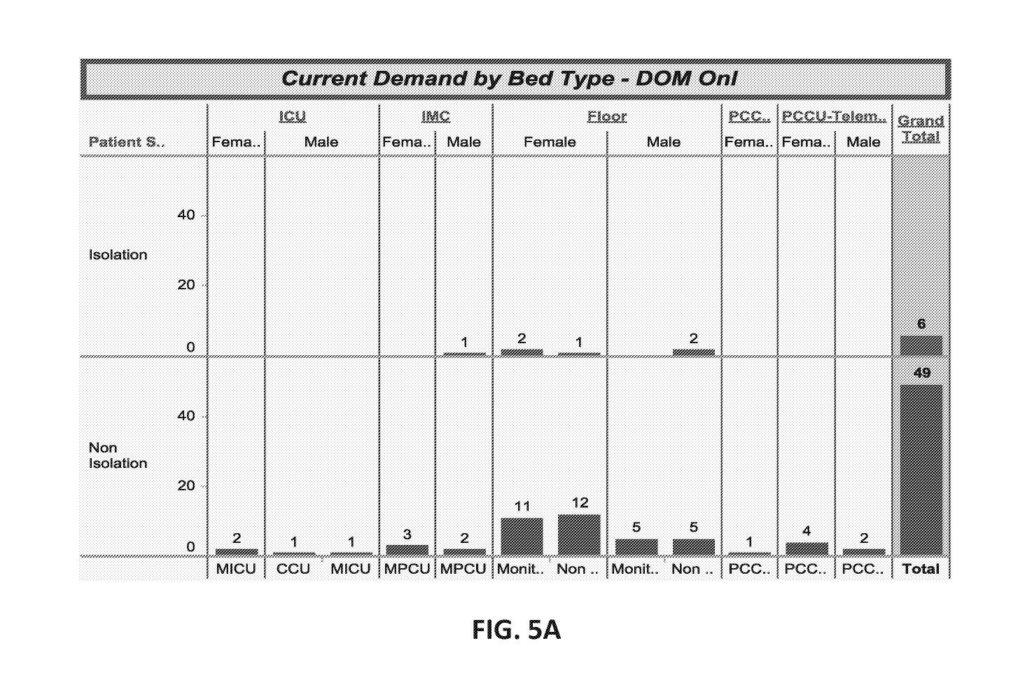

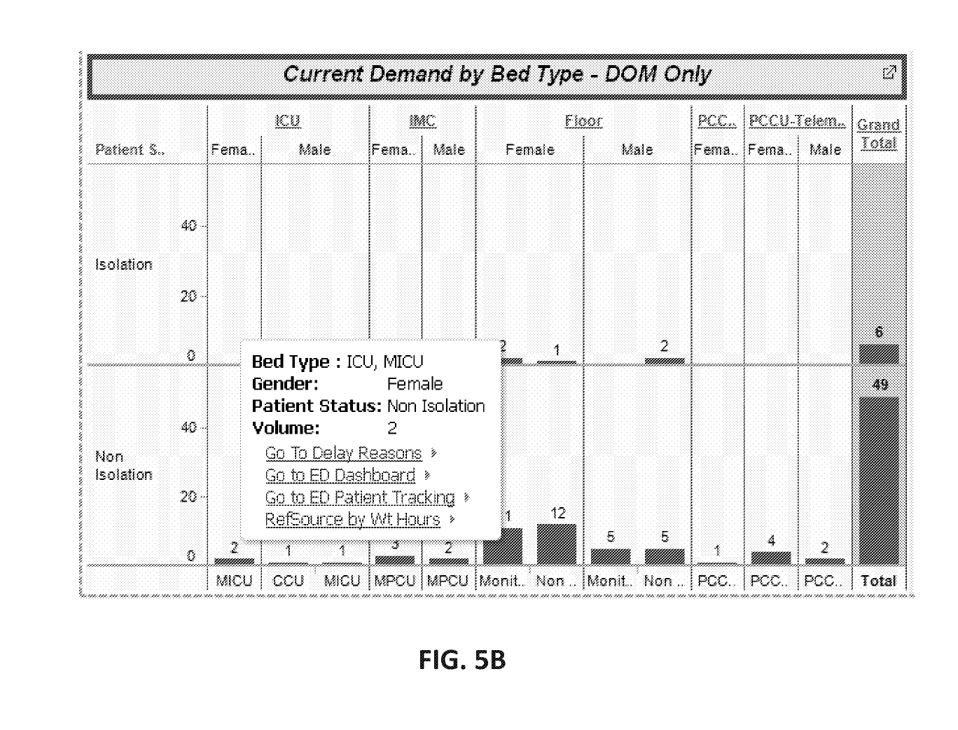

FIG. 5A illustrates an exemplary screen for the dashboard directed to current demand based on bed type, according to an embodiment of the present invention. The real-time demand can be further drilled down on gender, and special requirements. This view assesses how many isolation and non-isolation beds needed for female/male and monitored/non-monitored beds. These categories are represented by different colors for easy visuals. As illustrated in FIG. 5B, a drill down menu is available through the same screen to navigate through the entire dashboard. Users may choose to go to ED Dashboard to obtain more details about current situation in ED. There is an option of viewing this info at patient level and by wait hours. To track patient flow from ED to the in-patient bed, users may view ED patient tracking dashboard.

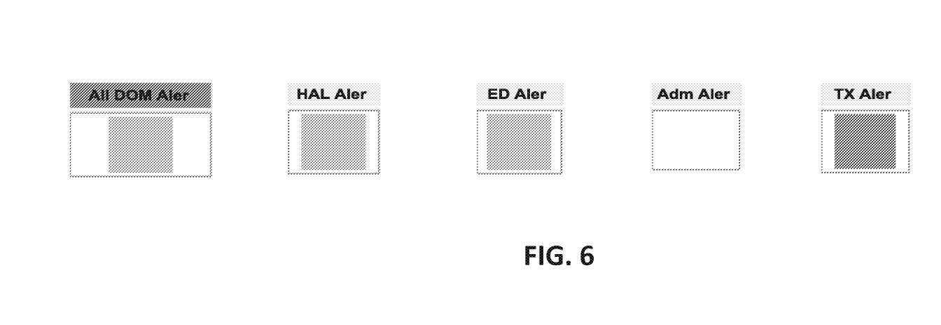

FIG. 6 illustrates an exemplary screen for the dashboard directed to a five-stage demand alert system, according to an embodiment of the present invention. The five stage alert system is available to a user to enhance the decision making process. It allows for assessment of the situation at a glance. Further information can then be sought, as needed, using the drill down graphical views. The referral sources listed in the alert system will change their color from a dark green for low alert to a red for high alert, as described with respect to the thumbnail of the five-stage alert system that appears on the home screen.

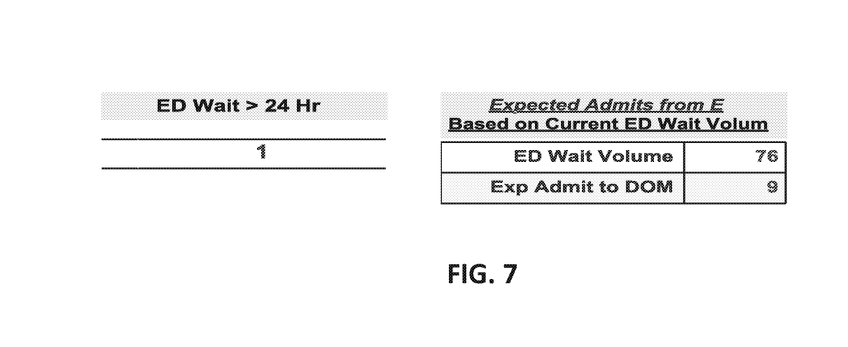

FIG. 7 illustrates an exemplary screen for the dashboard directed to a predictive demand from the ED, according to an embodiment of the present invention. This view allows users to identify any patients waiting in ED for more than 24 hours. This prompts appropriate staff to make a decision to contact ED and get status on those patients. If needed, send an Internal Medicine consultant to re-evaluate patient and decide further course of action. A predictive model is put together to alert our users of coming Inpatient volume from ED. This model is based on historical ED trend. Variables that predict expected admits are current ED load, historical % admits from total ED visits, and % admits to Medicine from total admits. Following is the calculation for projection:

Current Patients in ED*20%=Total IP admits.

Total IP admits*60%=Total Department of Medicine Admits

This prepares users in knowing what IP volume can be expected from ED in coming 3-4 hours and make plans accordingly.

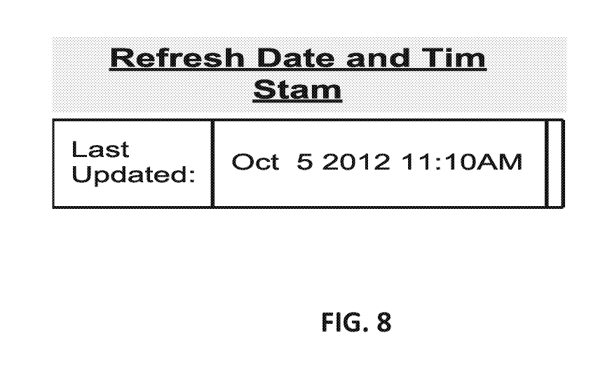

FIG. 8 illustrates an exemplary screen illustrating the update time and date for the last data update, according to an embodiment of the present invention. While the home screen displays the refresh date and time, there is also functionality included such that the user can manually refresh the data, in order to look at the most recent information. The user can refresh as many times as necessary during a session, especially because the bed and demand information can change rapidly during the decision making process.

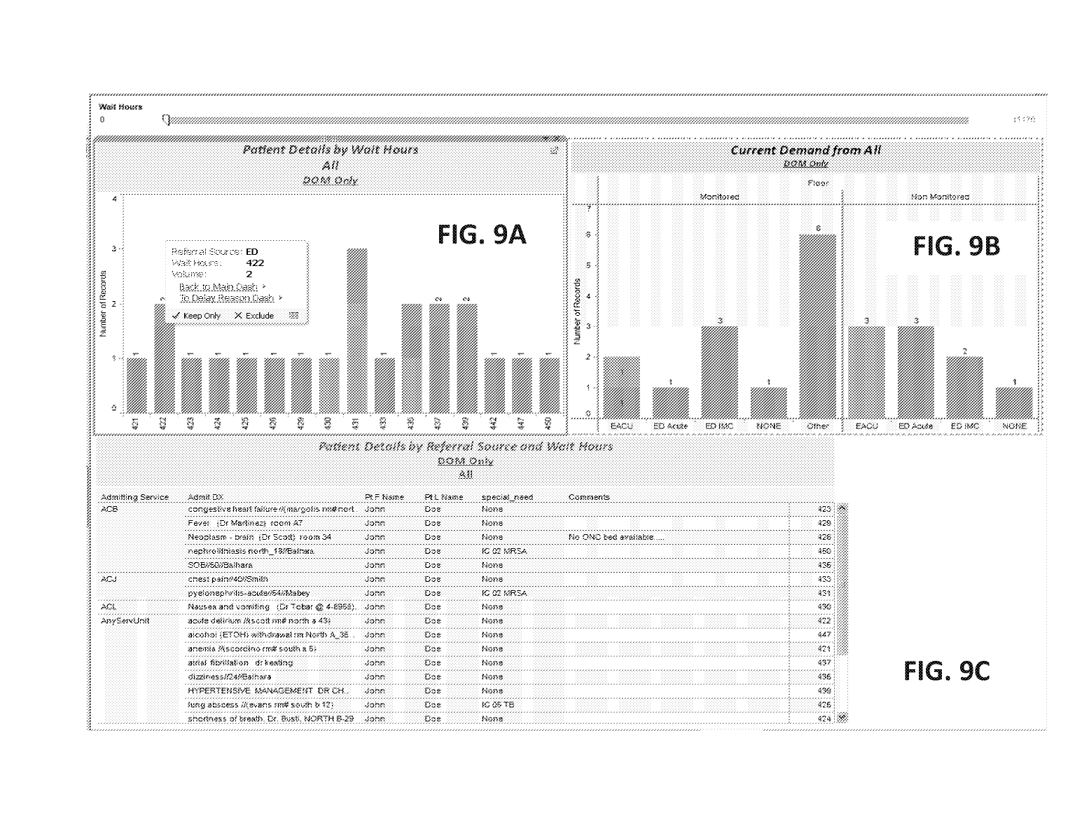

FIGS. 9A-9C illustrate exemplary screens with additional detail related to current demand, according to an embodiment of the present invention. As illustrated in FIGS. 9A-9C, Current demand can be drilled down and can be viewed by how long a patient has been waiting for the bed, which location he is currently waiting at, with other information such as admitting diagnosis, admitting service, admitting special requirements or any other comments associated with the patient. As illustrated in FIG. 9A, an option of filtering patient volume by wait hours allows focusing on a group that needs immediate attention.

Further with respect to FIG. 9A, the user can navigate back to Main Dashboard from this screen, as shown in the pop-up window. This view also allows filtering data on the same dashboard from first screen to isolate data points for better understanding. In the first screen at upper left hand side, each bar (X-Axis) represents total number of patients waiting. Y-Axis represents how long they have been waiting. This view enables users to determine volume by their total wait hours. The priority to admit the patients are based on severity and total wait hours. Users can use this screen to filter patient volume by wait hours to prioritize admissions.

As illustrated in FIG. 9B, the second screen shows where these patients are currently placed. In the example above, patients are categorized based on currently monitored or non-monitored status by locations in ED. Various bed types available in ED are EACU, Acute, IMC, etc. This screen helps in easy communication between the bed management team and ED, as users already know where to contact based on the units. It also helps in determining how critical the patient may be, for an example, patients admitted to ED EACU will be sicker (or needing extra attention) than the patients in ED Acute beds.

FIG. 9C illustrates patient level details. It breaks down patients by admitting service. Other details that users can view are admitting diagnosis, patient name, any special requirements (isolation status, sitters, psych beds, behavioral problems etc.), and other comments that may be useful for them to evaluate the need and assign the right type of beds to the patients. The numbers in last column is wait hours for the patient, how long the patient is waiting to get a bed.

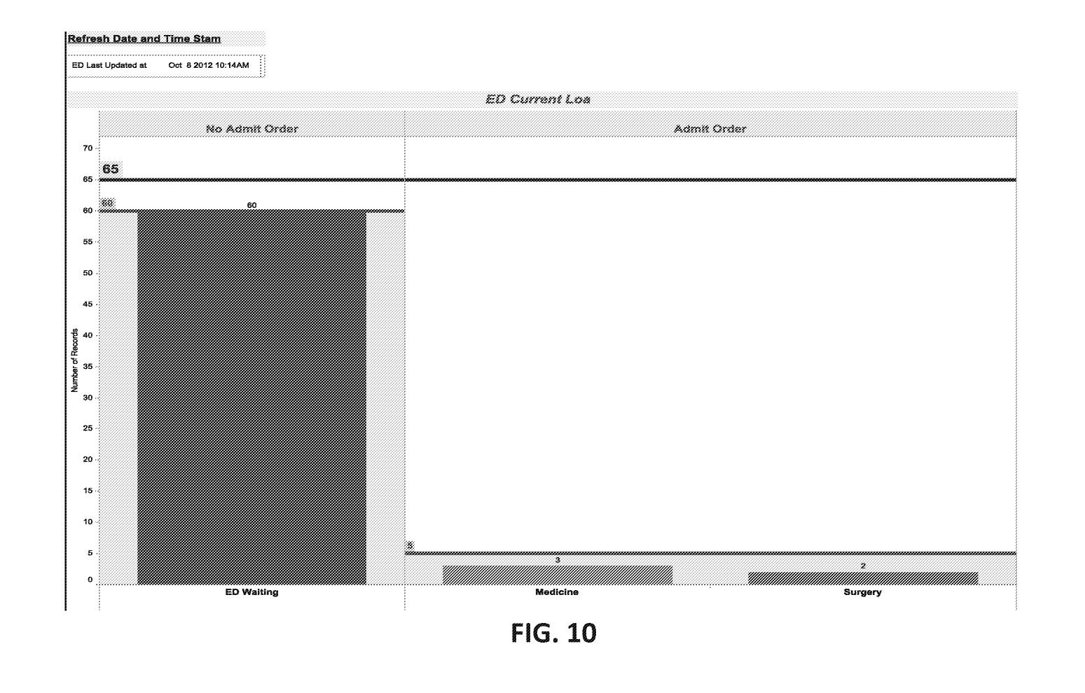

FIG. 10 illustrates an exemplary screen illustrating demand originating from the Emergency Department (ED), according to an embodiment of the present invention. This view shows current ED Volume Load. This dashboard can be accessed either by clicking one of the tabs or by using navigation link from the main dashboard (or any other dashboards where this option is available). The blue line represents total patients waiting in ED. This volume is further divided into two categories of patients, one with Admit Orders (decision is taken to admit the patients by ED Attending) and the other Without Admit Order (patients that are still getting evaluated and treated before a decision of admission is made). Pink line is total volume for each of these categories. Patients with admit order is further broken down by department and service to which they need admission. A drill down to patient level details is available through this screen. This screen also allows navigation between different dashboards such as Main Dashboard, Delay reasons dashboard etc.

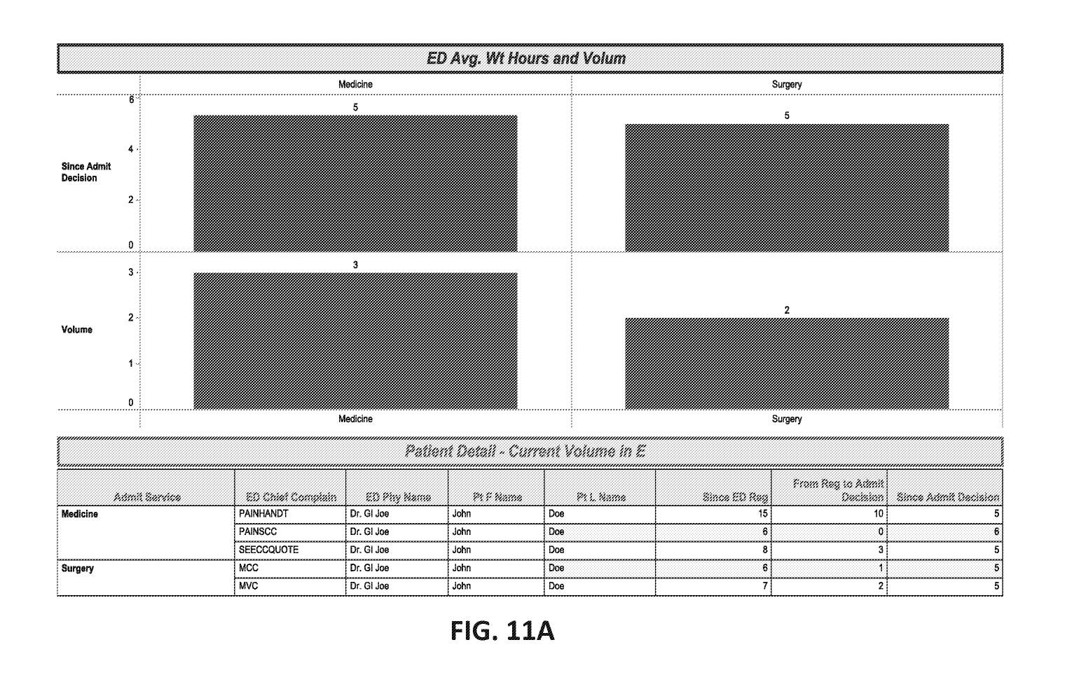

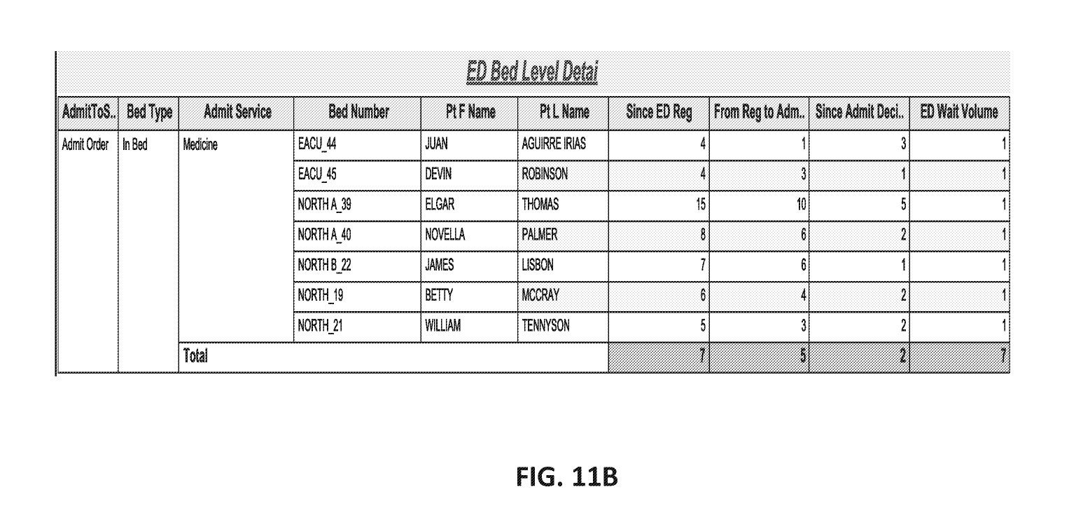

FIGS. 11A and 11B illustrate exemplary screens accessible from the screen illustrated in FIG. 10 and illustrating ED demand details, according to an embodiment of the present invention. As illustrated in FIG. 11A, from the previous screen, illustrated in FIG. 10, users can drill down to patient level information by clicking on appropriate options. For example, a user drills down on Admit Orders categories. This action will take him to the next dashboard that has patient level details. This dashboard allows graphical as well tabular view for users to assess the status of the patient in ED. This screen shows how many patients are waiting to be admitted by departments/service (red bars) and what is their average waiting time in hours (blue bars). The table below the graph shows patient level details and breaks down wait time into three periods. The table shows, by departments/services at patient level, who is the ED attending for the patient and what is patient's chief complain. It also tracks patient's time line from the moment he was received at ED register to the point the decision was taken to admit him. “Since ED Reg.” shows how long it is been since the patient was received at ED Register. The second column, “From Reg. to Admit Decision” shows how long did it take for ED Attending to take a decision to admit the patient after he was registered. The last column represents how long it is been since a decision to admit is made and patient is waiting for an inpatient bed. The users can look at this view to prioritize the bed assignment by looking at different variables. As illustrated in FIG. 11B, this view can be further drill down at bed level details. It shows users which bed the patient is currently admitted and since how long.

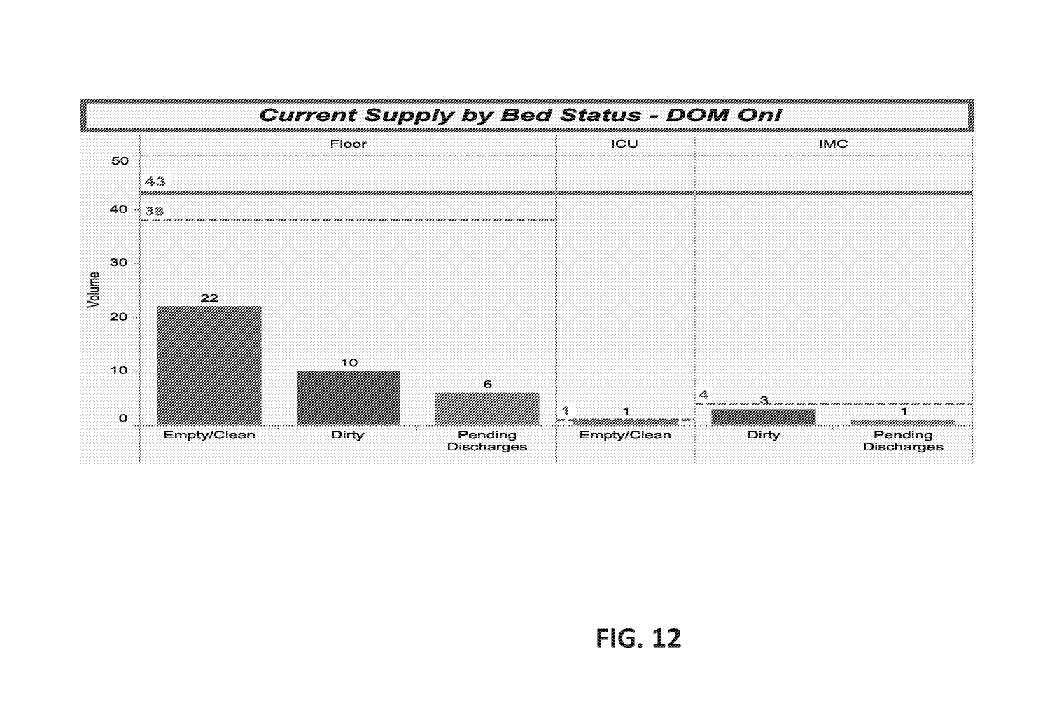

FIG. 12 illustrates an exemplary screen directed to current supply of beds, according to an embodiment of the present invention. The current supplies of beds are viewed on this screen. This view shows currently available (Empty/Clean) and shortly available beds (Dirty, Pending Discharges and Deaths). This supply can be further broken down by bed type, namely, Floor, IMC and ICU beds. Each of these categories is summed to show total available beds by each bed type. A drill down option is available to further analyze it at unit/location and bed levels. Each bed status is color coded for easy visualization. This screen shows current bed status at unit level that is broken down by bed types. It shows, by unit, how many beds are empty/clean, dirty, on hold, closed, out of service, occupied and occupied isolation. The last column shows the total beds that the unit has (includes closed and out of service beds). This view helps in assessing available staffed and open beds, and beds that can be made available in time of crisis. A drill down option from this view will display bed level information as show in the following figure.

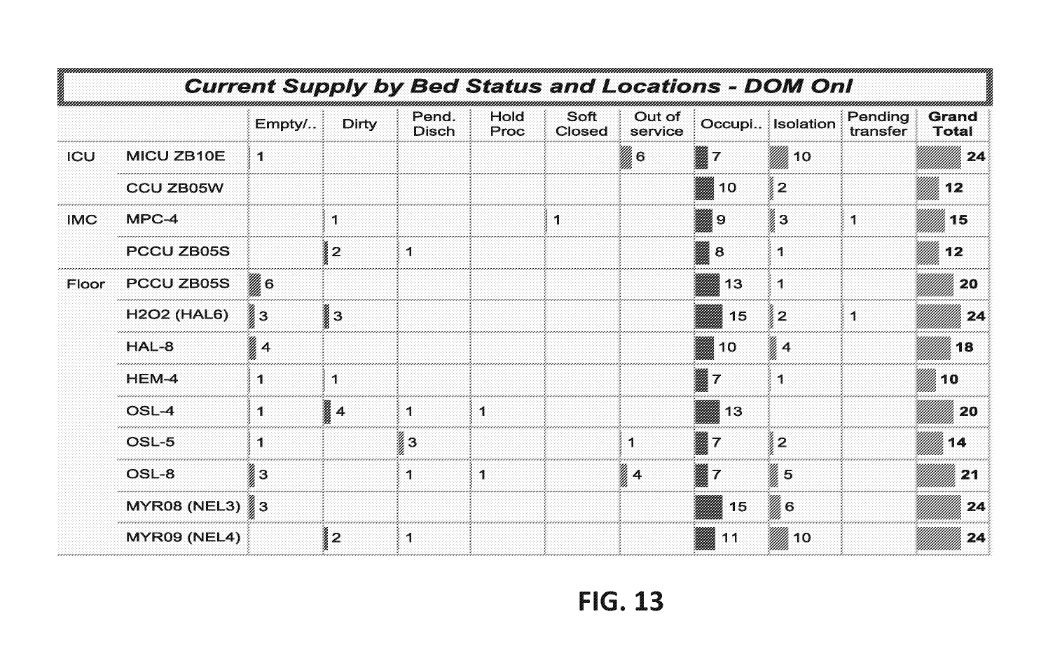

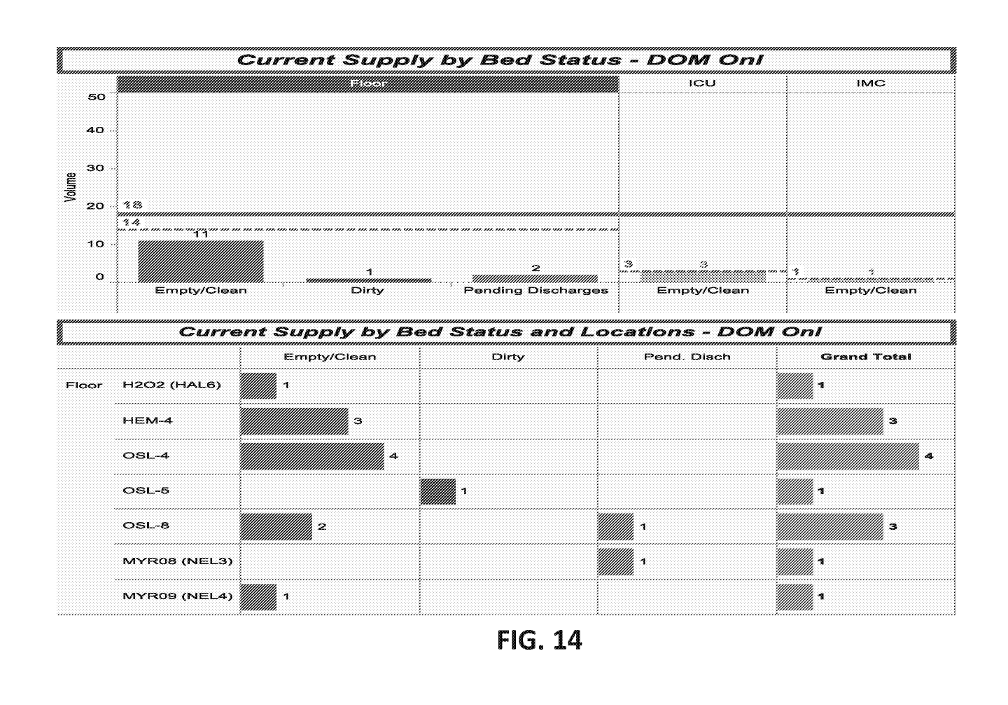

FIG. 13 illustrates an exemplary screen directed to details of current bed supply, according to an embodiment of the present invention, and FIG. 14 illustrates a drill down screen with more narrow details of bed supply based on those shown in FIG. 13. This screen shows current bed status at unit level that is broken down by bed types. It shows, by unit, how many beds are empty/clean, dirty, on hold, closed, out of service, occupied and occupied isolation. The last column shows the total beds that unit has (includes closed and out of service beds). This view helps in assessing available staffed and open beds, and beds that can be made available in time of crisis. A drill down option from this view will display bed level information as show in the following figure. A drill down option is also available through first supply screen to narrow down beds on the second supply screen. The drill down screen shows by unit total number of beds available. Bed status is color coded to visually identify them.

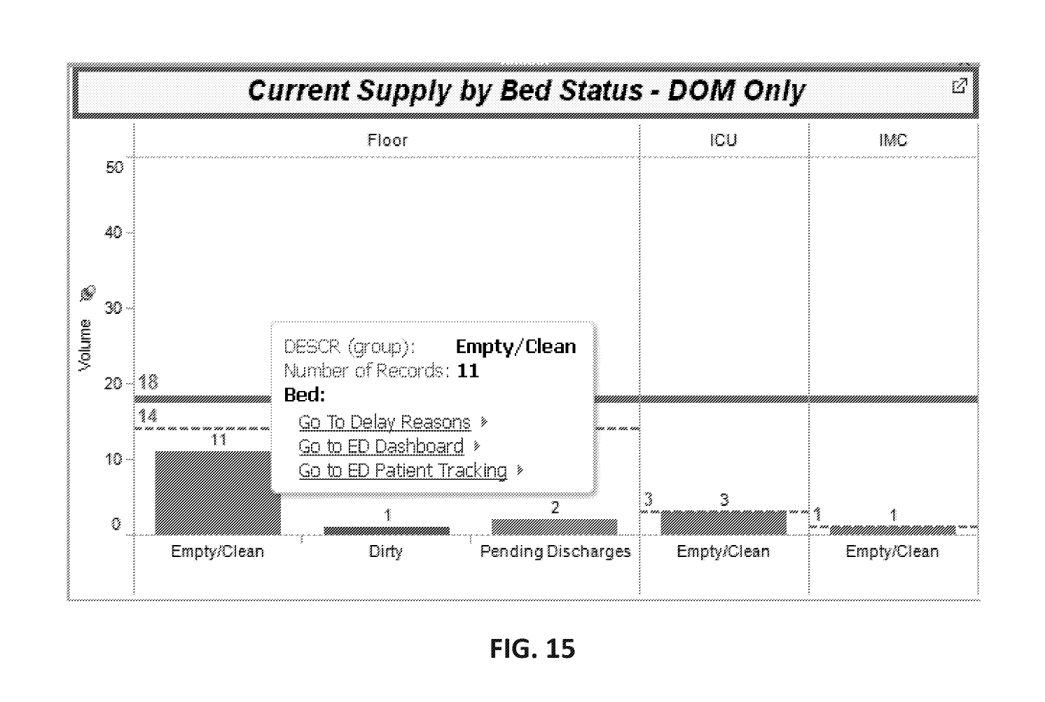

FIG. 15 illustrates an exemplary screen related to discharge details, according to an embodiment of the present invention. From the main Supply screen, a navigation option is available to further view potential discharges that can take place. “Delay Reasons” gives users further insight as how many patients are medically ready to go home, however occupying inpatient beds for some reasons. The details of Delay Reasons are in the following screens.

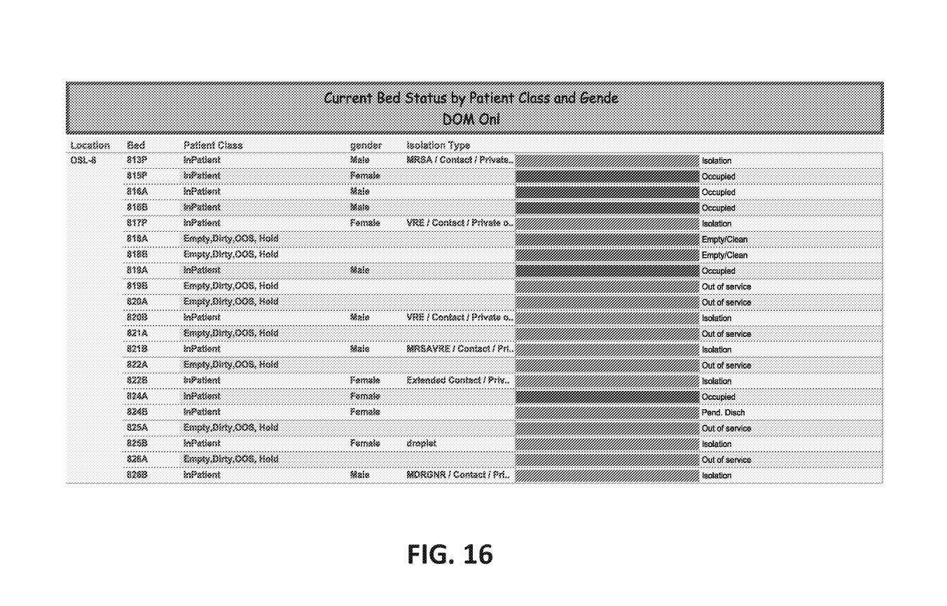

FIG. 16 illustrates an exemplary screen directed to details of bed status, according to an embodiment of the present invention. This screen can be accessed by clicking on units or bed type on the previous screen. This shows bed status at unit and bed levels. It displays, by unit and bed numbers status of the beds (empty/clean, dirty, on hold, closed, out of service, occupied and occupied isolation), patient class (inpatient or outpatient), patient gender, and isolation type if any. This allows users to easily scan for available beds to cohort patients based on gender or isolation. Each bed is color coded based on their status.

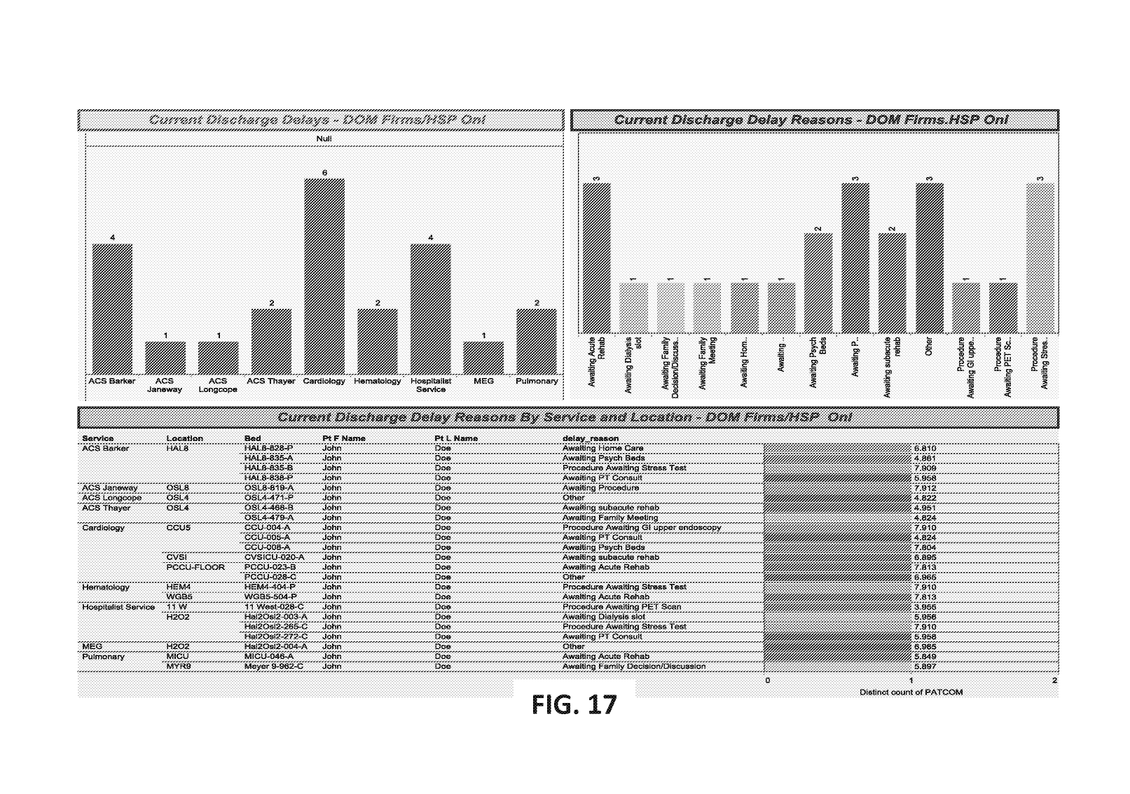

FIG. 17 illustrates an exemplary screen directed to details of discharge delay, according to an embodiment of the present invention. The Delay Discharge Reason screen shows, by admitting service, how many patients are medically ready to go home and reasons associated with their delays in discharges. These patients are exclusive of pending discharge volume. This information can be viewed at unit level, bed level and patient level details. The patient level details view shows days since the patient is waiting and occupying a bed. This helps users to prioritize tasks and ask questions. Based on this information it helps users to take necessary actions to get a process started to free up a bed by meeting the need of the patient. Each reason is color coded for visual identification.

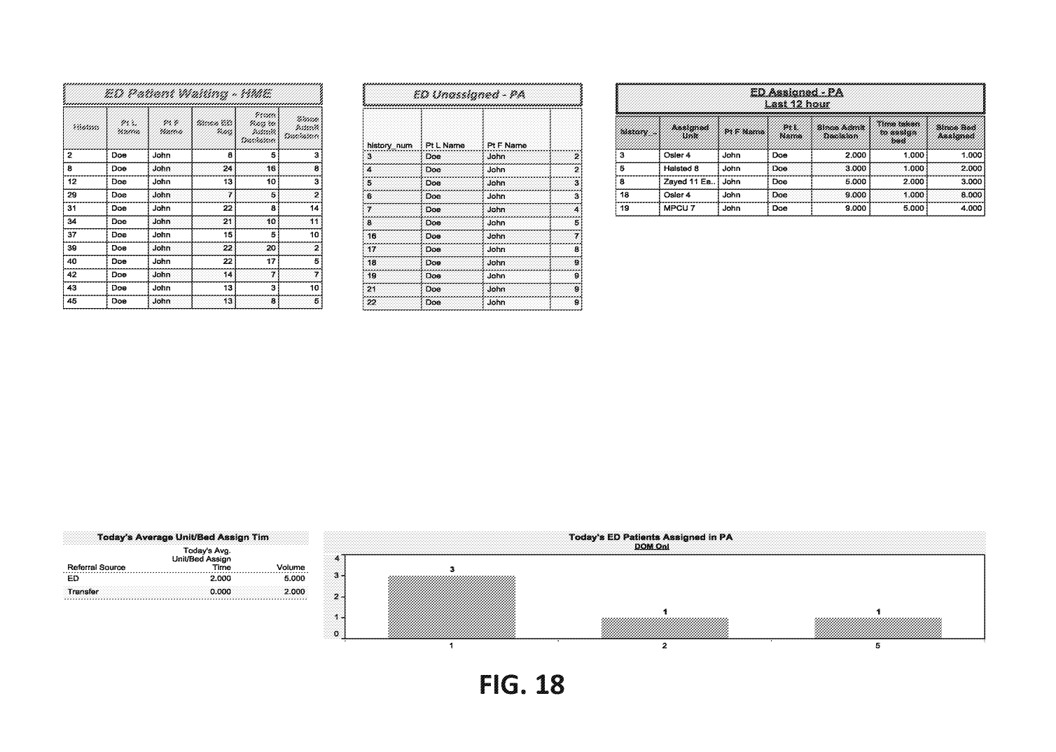

FIG. 18 illustrates an exemplary screen for tracking patient movement, according to an embodiment of the present invention. ED Patient Tracking dashboard tracks patient's movement that arrived at ED and needed an inpatient bed. This screen shows a timeline of a patient's movement from ED to Medicine Beds. Time line includes: Time since patients walked-in in ED, to the time decision was taken to admit him and finally time it took to assign a bed to the patient. This allows capturing the total patient throughput time at patient level for the Department of Medicine. It also shows current average times it take to assign a unit/bed by referral source. This information helps every stakeholder to prioritize their efforts to free up a bed and identify any issues in the entire patient throughput process that might be delaying placement of the patient in the bed. Some of the issues that might be affecting patient throughput are in the following screens.

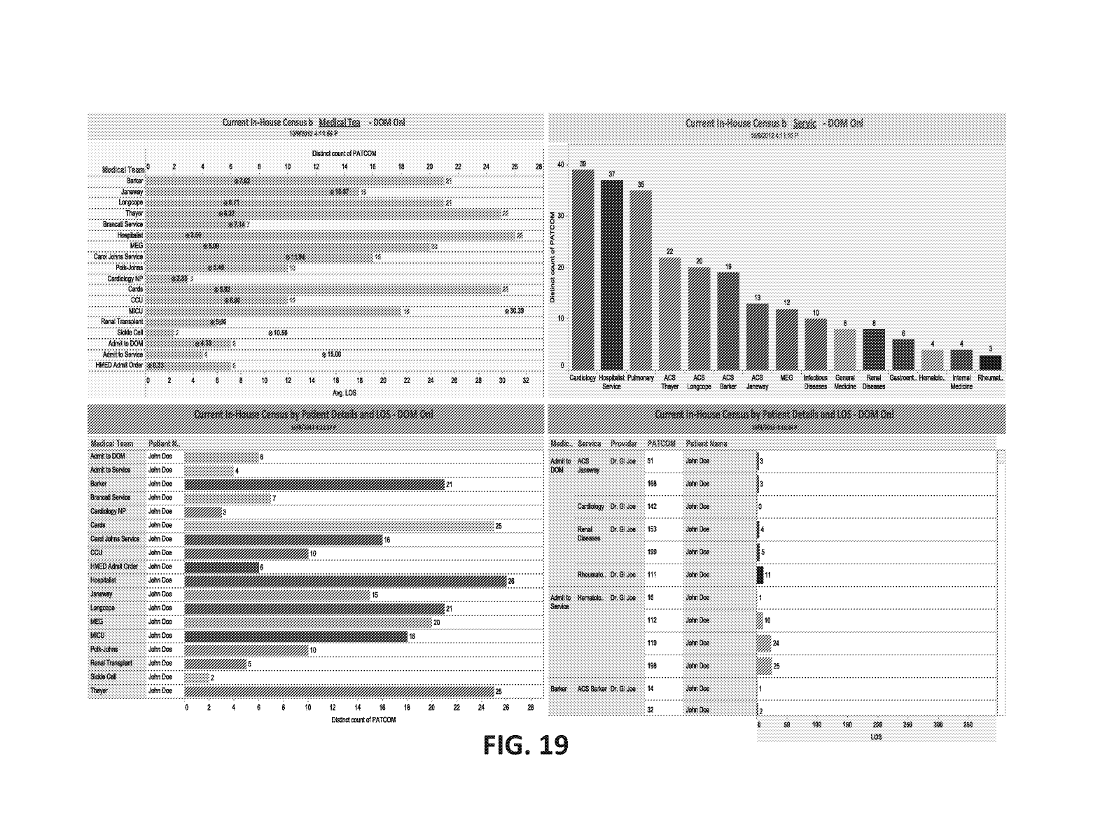

FIG. 19 illustrates an exemplary screen for showing impediments to patient throughput, according to an embodiment of the present invention. This dashboard gives an overview of current census of the department of medicine and ALOS for the patients that are currently occupying beds. The first screen on upper left corner shows census by medical team. Most medical teams can have only 30 or less patients at a given point of time. If the team is capped then the patient admit wait time could be extended unless a patient is discharged by the team. ALOS for medical team helps understand what the turnaround rate of patients is. From this screen the data can be drilled down on medical team to view patient level details by medical service and locations.

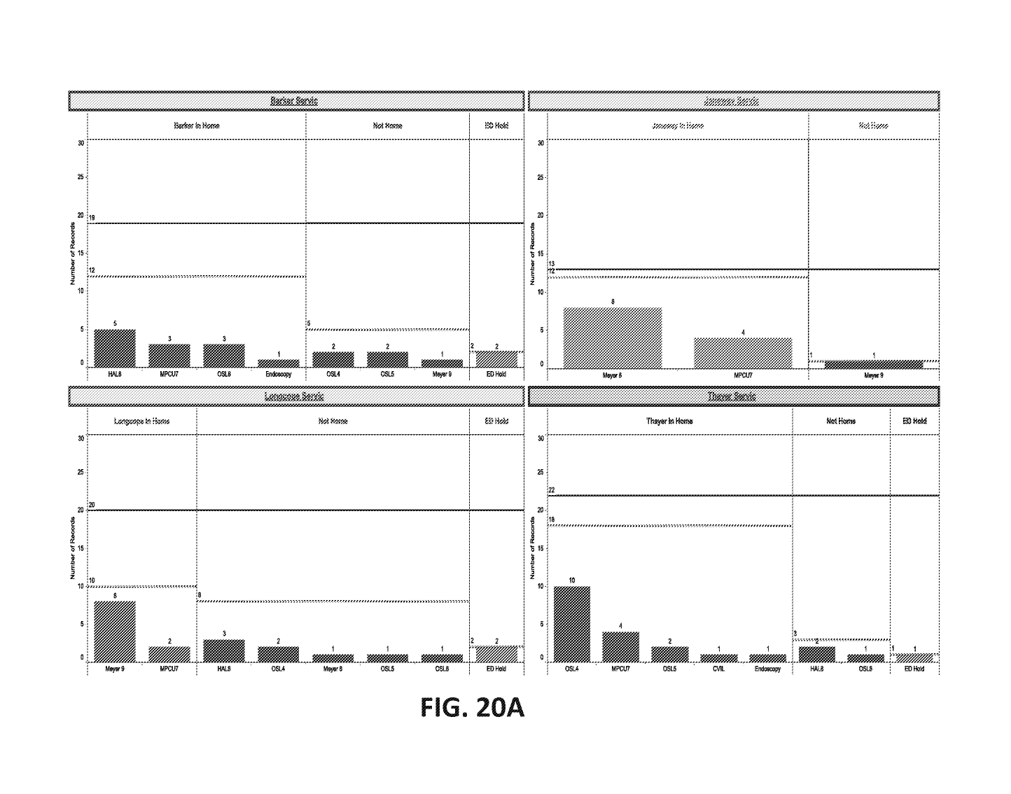

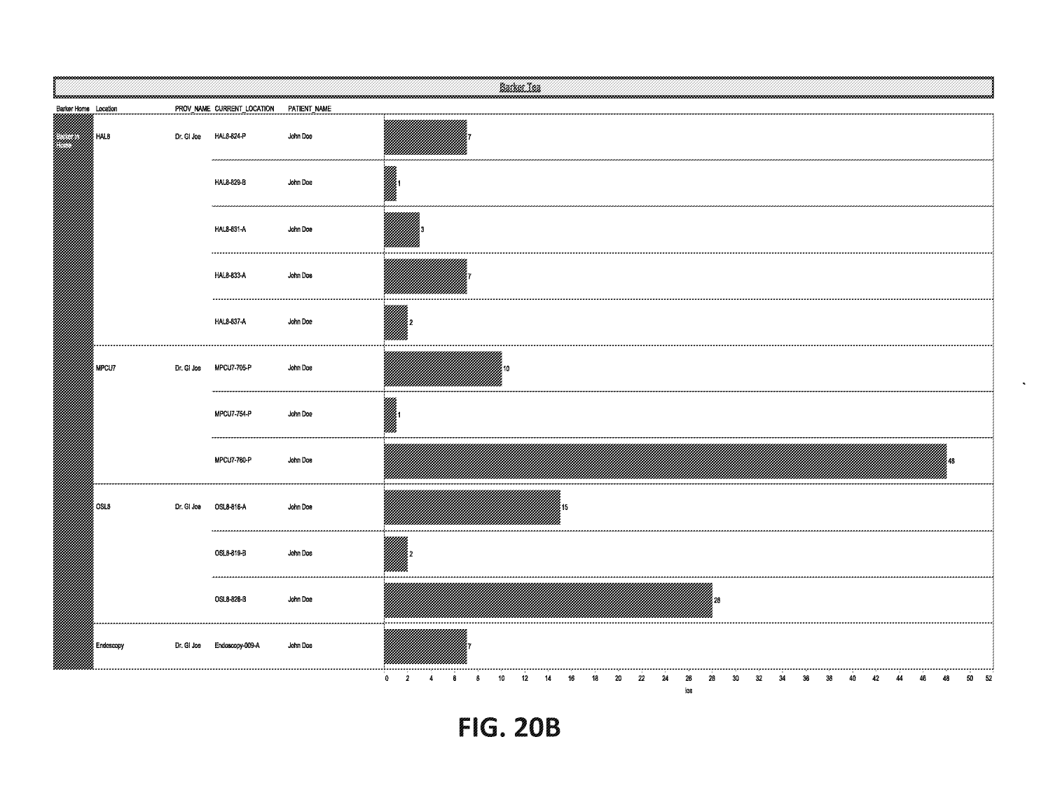

FIGS. 20A and 20B illustrate exemplary screens directed to medical team location geography, according to an embodiment of the present invention. For better, faster and efficient care coordination, the patients are placed according to medical team location geography. Each medical team is assigned a location. If the bed is not available in the desired location for the patient, then patient admit wait hour may be extended until a bed frees up in the location. As illustrated in FIG. 20A, this screen categorizes each location into Home Location, Not Home Location and ED Hold. Each team has different colors for Home Location volume. For Not Home Location and ED Hold volume colors are the same for all the team (blue for Not Home Location and orange for ED Hold patients). This volume is summed up by these categories to quickly assess percent of patients in location or not in location. As illustrated in FIG. 20B, this information can be drilled down to the patient level by location and bed. This facilitates movement of patients to place them in the right/appropriate geography to achieve effective and quick patient throughput.

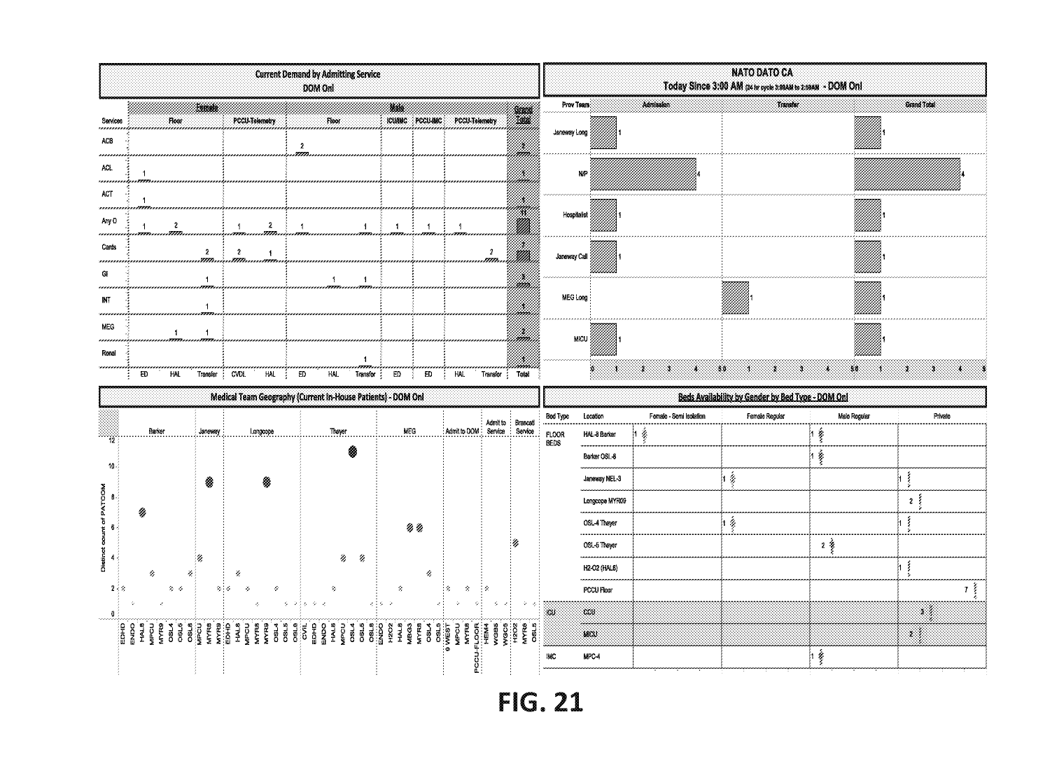

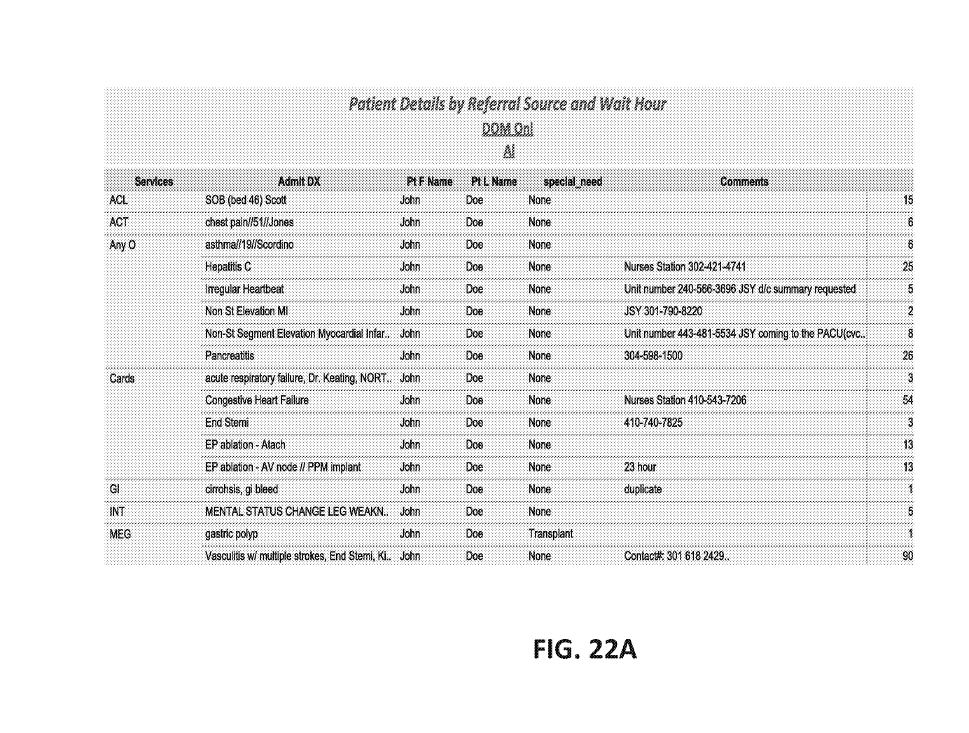

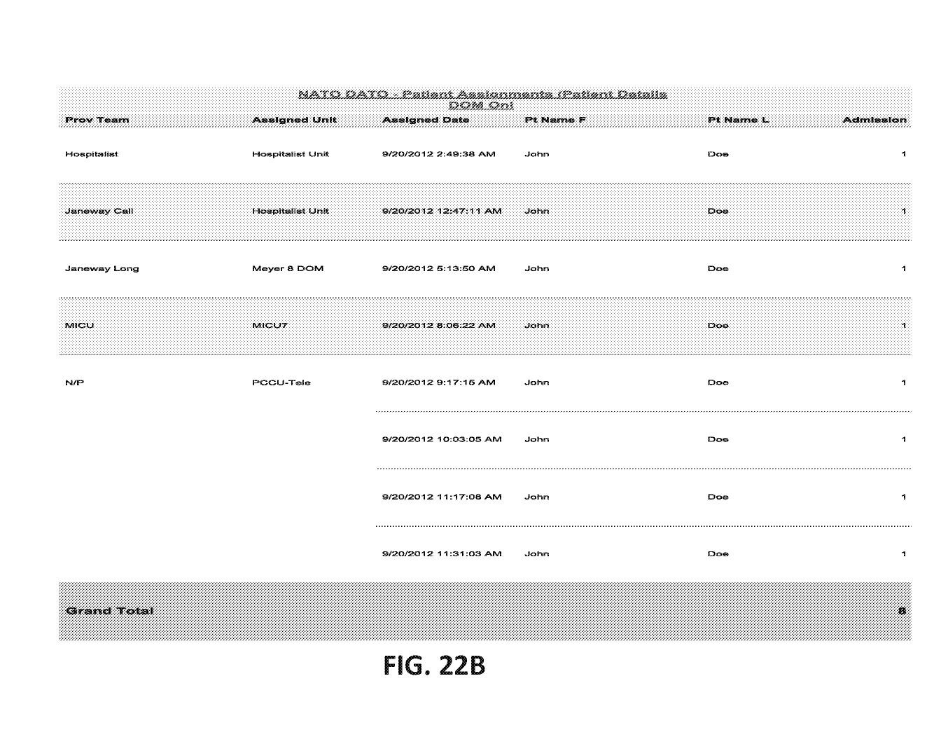

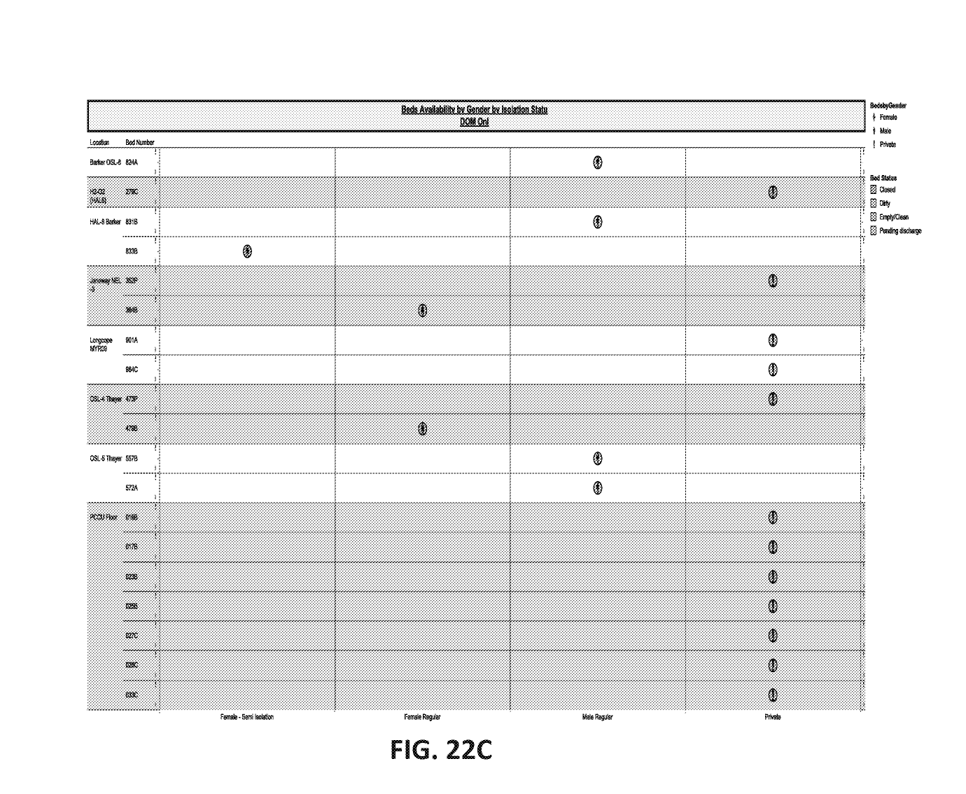

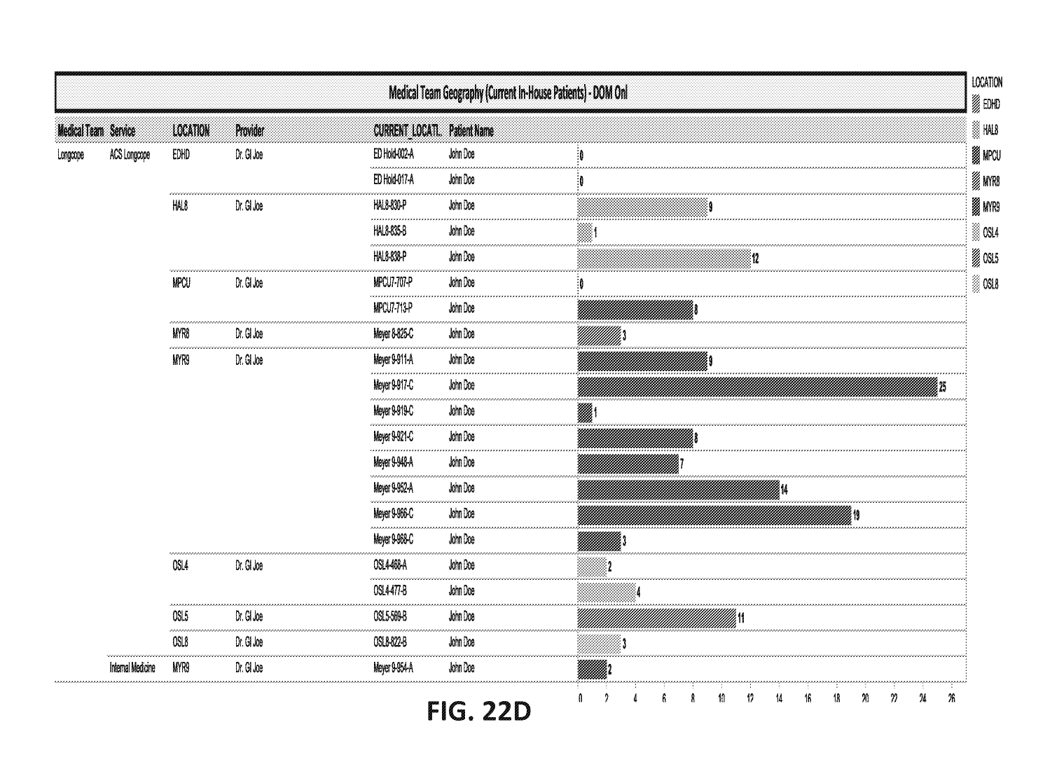

FIG. 21 illustrates an exemplary screen detailing admission capacity, and FIGS. 22A-22D illustrate exemplary screens showing admission capacity details, according to an embodiment of the present invention. This dashboard helps to identify any capacity issues by residents and inters to admit the patients. FIG. 22A shows patients waiting for beds by admitting service. Each admitting service has residents and interns who admit patients. FIG. 22B shows by resident and intern teams (each team is dedicated to different services and names and classification are internally defined by the hospital or other regulating group) total patients admitted during their shift. If teams are capped and the patients are still waiting to be admitted (based on admitting service) then patients' admit wait hours may be extended. FIG. 22C shows team capacity and geography (as discussed earlier) in a graphical format (bigger dots mean more volume) by location. FIG. 22D shows available beds (empty/clean, dirty, pending discharge and death) for male, female based on their isolation status. Female Isolation bed is shown with Female Graphic in pink under Female-Isolation category. This means that there is a bed available for female that may be in need of isolation bed (based on special needs in demand screen) and likewise. A drill down option to patient level details is available through each of these screens.

It should be noted that although these exemplary screens were included in the description of the present invention the invention is not limited to these screens and options. Any other suitable screens or detail options known to or conceivable by one of skill in the art could also be used. The screens can also appear in any order and can be linked to one another through click links, menus, or a search function, or any other way known to or conceivable by one of skill in the art.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A system for presenting availability and demand for hospital beds within a department of medicine to a user comprising:
a server configured to store data related to hospital bed availability and hospital bed demand;
a computing device configured to communicate with the server, said computing device further in communication with a non-transitory computer readable medium being programmed to execute steps comprising:
tracking a patient from a point of entry of the patient to a point of hospital bed occupation to determine demand for the hospital bed;
tracking hospital bed availability within the department of medicine; and
displaying the demand and availability for the hospital beds to the user.

2. The system of claim 1 further comprising creating a network between the server and the computing device.

3. The system of claim 1 further comprising a user interface configured for the user to input information into the system as well as configured for the user to interact with the system.

4. The system of claim 1 wherein the non-transitory computer readable medium is further programmed to execute a step comprising tracking demand for the hospital beds from different points of admission.

5. The system of claim 4 wherein the different points of admission are color coded.

6. The system of claim 1 wherein the non-transferable computer readable medium is further programmed to execute a step comprising displaying the demand and availability for the hospital beds categorized by a level of care needed by the patient.

7. The system of claim 1 wherein the non-transferable computer readable medium is further programmed to execute a step comprising alerting the user to high levels of congestion in hospital bed availability and hospital bed demand.

8. The system of claim 7 wherein the alerting the user is executed using a color coded system indicating a range from high alert to low alert.

9. The system of claim 1 wherein the non-transferable computer readable medium is further programmed to execute a step comprising allowing the user to select detailed views of hospital bed availability and hospital bed demand based on specific criteria selected by the user.

10. A method for presenting availability and demand for hospital beds within a department of medicine to a user comprising:
using a computing device configured to communicate with a server configured to store data related to hospital bed availability and hospital bed demand;
having the computing device communicate with a non-transferable computer readable medium programmed for:
tracking a patient from a point of entry of the patient to a point of hospital bed occupation to determine demand for the hospital bed;
tracking hospital bed availability within the department of medicine; and
displaying the demand and availability for the hospital beds to the user.

11. The method of claim 10 wherein the non-transferable computer readable medium is further programmed for creating a network between the server and the computing device.

12. The method of claim 10 wherein the non-transferable computer readable medium is further programmed for configuring a user interface for the user to input information into the system as well as configured for the user to interact with the system.

13. The method of claim 10 wherein the non-transferable computer readable medium is further programmed for tracking demand for the hospital beds from different points of admission.

14. The method of claim 13 wherein the non-transferable computer readable medium is further programmed for color coding the different points of admission.

15. The method of claim 10 wherein the non-transferable computer readable medium is further programmed for displaying the demand and availability for the hospital beds categorized by a level of care needed by the patient.

16. The method of claim 10 wherein the non-transferable computer readable medium is further programmed for alerting the user to high levels of congestion in hospital bed availability and hospital bed demand.

17. The method of claim 16 wherein the non-transferable computer readable medium is further programmed for alerting the user is executed using a color coded system indicating a range from high alert to low alert.

18. The method of claim 10 wherein the non-transferable computer readable medium is further programmed for allowing the user to select detailed views of hospital bed availability and hospital bed demand based on specific criteria selected by the user.

19. The method of claim 10 wherein the non-transferable computer readable medium is further programmed for displaying a number of screens with information through which the user can toggle.

20. The method of claim 10 wherein the non-transferable computer readable medium is further programmed for displaying information classified by a category, wherein the category comprises one selected from a group consisting of patient volume, bed availability, bed location, demand origin, bed status, discharge delay, patient movement, impediments to patient movement, medical team location, and admissions capacity.
